(19) United States
(12) Patent Application Publication
(10) Pub. No.: US 2013/0115302 A1
(43) Pub. Date: May 9, 2013

(54) POLYSACCHARIDE MICRO-PARTICLE ENCAPSULATIN GROWTH FACTOR

(71) Applicant: Le Shin CHANG, Taoyuan City (TW)

(72) Inventor: Le Shin CHANG, Taoyuan City (TW)

(73) Assignee: MICORNA DERMAL THERAPY LAB CO., LTD., Taipei City (TW)

(21) Appl. No.: 13/439,128

(22) Filed: Apr. 4, 2012

(30) Foreign Application Priority Data
Nov. 8, 2011 (TW) 100140777

(51) Int. Cl. A61K 38/18; A61K 9/14; B82Y 5/00

(52) U.S. Cl. 424/499; 514/7.6; 514/9.6; 514/9.1; 514/8.2; 977/773; 977/906

(57) ABSTRACT

A polysaccharide micro-particle encapsulating a growth factor is disclosed and shall include one or more growth factors, and a polysaccharide shell forming a space to encapsulate the growth factor by electrostatic interaction. Also, a method for manufacturing a polysaccharide micro-particle encapsulating a growth factor is disclosed, which shall include the following process: (A) providing a pH 4.6-6 polysaccharide solution and a growth factor; and (B) adding the growth factor to the polysaccharide solution, and adjusting the polysaccharide solution to a pH of 6-8 to obtain the polysaccharide micro-particle encapsulating the growth factor by electrostatic interaction. According to the polysaccharide shell structure, the growth factor can be stored for a long period of time and heal skin wounds, mucositis, and corneal ulcer effectively.

### CROSS REFERENCE TO RELATED APPLICATION

This application claims the benefits of the Taiwan Patent Application Serial Number 100140777, filed on Nov. 8, 2011, the subject matter of which is incorporated herein by reference.

### BACKGROUND OF THE INVENTION

1. Field of the Invention

The present invention relates to a polysaccharide micro-particle and, more particularly, to a polysaccharide micro-particle suitable for directly encapsulating a growth factor by electrostatic interaction and conformational changes of polysaccharide. Hence, the encapsulated growth factor can be stabilized and stored for a long period of time and have increased efficacy for healing skin wounds.

2. Description of Related Art

Growth factors are found in platelets, macrophagocytes, and monocytes, which are essential polypeptides to human bodies and classified into various species such as epidermal growth factor, fibroblast growth factor, platelet-derived growth factor, and nerve growth factor. Such growth factors are able to stimulate cell growth, differentiation, proliferation, and further induction of stem cells differentiating into mature tissues, and thus are considerably valuable in medical treatments. With the development of science and technology, mass production of a specific growth factor with high purity is commercially achievable by current clone techniques, resulting in considerable decrease in costs for these growth factors. Therefore, growth factors can be applied to tissue regeneration, for example, in the treatment of chronic wounds of diabetes, mucositis, scalds, and corneal restoration, as well as skin protection and anti-wrinkles.

However, growth factors are unstable in an aqueous solution. In order to keep efficacy of growth factors and prevent degradation, they are made by lyophilization and stored at −20° C. When someone wants to use growth factor lyophilized powder, it is taken out of the −20° C. environment and readily formulated into a solution with a desired concentration convenient for clinical use. Because of hydrogen bonding interaction, it is easy for amino groups to leave from growth factors in an aqueous solution. This results in conformational changes in growth factors and makes them lose binding affinity to corresponding receptors. When growth factor lyophilized powder is formulated into various medical preparations, growth factors can be kept active at room temperature for 7 days only. Even if the preparations are stored under refrigeration, the activity of growth factors can only be maintained for three months. However, the time period from manufacturing preparations until the preparations are purchased is often more than several months, even up to one year or more.

Hence, the loss of activity in preparations is currently a major problem for commercializing growth factors.

In current techniques, U.S. Pat. No. 7,901,711B1 describes that negatively charged PGA cores that are conjugated to positively charged insulin or growth factors are encapsulated with chitosan. Thus, pharmaceutical carriers encapsulating insulin or growth factors can be formed and kept stable for 6 months. However, the method described in the above-mentioned patent is complicated and also increases production cost. Besides, PGA does not act as enzymes and may cause allergic reaction in users, and the storage term of the pharmaceutical carriers in the above-mentioned patent is still shorter than those of common drugs. Thus, it is not sufficient to satisfy-consumers' demands for commercialization of the pharmaceutical carriers. Conventional techniques show that silica and chitosan are used in encapsulating growth factors extracted from young fish, but does not further describe the storage term of growth factors. In addition, silica may also cause allergic reactions.

In view of the above, long-term activity of growth factors in preparations, biocompatible, biodegradable, nontoxic, and production costs of carriers are required in the commercialization of growth factors. If growth factors with long-term activity can be encapsulated in carriers with low biocompatible, biodegradable, nontoxic, and production costs, it will benefit for patients who suffering from chronic wounds.

### SUMMARY OF THE INVENTION

The objective of the present invention aims to provide a polysaccharide micro-particle encapsulating growth factors. Polysaccharide encapsulation can stabilize the activity of growth factors, prolong storage life thereof, and promote its efficacy for healing wounds.

Another objective of the present invention aims to provide a method for manufacturing a polysaccharide micro-particle encapsulating growth factors to maintain the activity of growth factors. The conformation, charge distribution, and size of polysaccharide particles can be controlled by adjusting pH value, thus allowing the polysaccharide to encapsulate growth factors via electrostatic interaction. In addition, polysaccharide is able to encapsulate one or more growth factors optimizing the treatment for wounds of various severity.

To achieve the objectives mentioned above, the present invention shall provide a polysaccharide micro-particle that can encapsulate growth factors including one or more growth factors; and a polysaccharide shell forming a space to encapsulate growth factor. The charge of the polysaccharide shell is positive and that of the growth factor is negative in a neutral solution, thus the polysaccharide shell is able to encapsulate the growth factor by electrostatic interaction.

The growth factor encapsulated by the polysaccharide micro-particle mentioned in the above-mentioned paragraphs can be acidic or alkaline growth factors. The following are desirable growth factors: epidermal growth factor (EGF), recombinant human epidermal growth factor (rhEGF), fibroblast growth factor (FGF), and platelet-derived growth factor (PDGF). The fibroblast growth factor (FGF), recombinant human epidermal growth factor (rhEGF) or a combination of any of these are more desirable growth factors.

The polysaccharide shell can be made of any material and all polysaccharides can be used as the polysaccharide shell of the present invention. Desirable polysaccharide shells are made of at least one of the following materials: chitin, chitosan, mucopolysaccharide, cellulose, starch, and peptidoglycan. Polysaccharide shells made of chitosan, mucopolysaccharide, or a combination of these are more desirable.

The polysaccharide particle of the present invention can be of any size, preferably less than 3,000 nm; size between 1-1,000 nm is more favorable; size between 1-500 nm is even better, and size between 30-200 nm is the best to achieve best efficacy for healing wounds.

Isoelectric points of various growth factors can be found over both acidic and alkaline pH ranges. Hence, the present invention provides respective methods for manufacturing polysaccharide micro-particles encapsulating growth factors with acidic and alkaline isoelectric points. These methods are classified under the sol-gel method. For growth factors with acidic isoelectric point, the present invention provides a method for manufacturing a polysaccharide micro-particle encapsulating a growth factor in the following process: (A) providing a polysaccharide solution and one or more growth factors, wherein the polysaccharide solution has a pH of 4.6-6; and (B) adding the growth factor to the polysaccharide solution and adjusting the pH of the polysaccharide solution to 6-8, preferably between 6.5-7.4, to form the polysaccharide micro-particle encapsulating the growth factor, according to the present invention, by electrostatic interaction. If the isoelectric point of the growth factor is acidic (such as EGF with isoelectric point of pH 4.6 and acidic FGF with isoelectric point of pH 5.6), the growth factor is negatively charged and the polysaccharide is positively charged in the pH 4.6-6 polysaccharide solution. When the pH value of the polysaccharide solution is adjusted to neutral or slightly alkaline, the polysaccharide will encapsulate the growth factory by conformation using electrostatic interaction, forming the polysaccharide micro-particle encapsulating the growth factor according to the present invention.

Because most polysaccharides are insoluble, long-chain large molecules, it is difficult for them to encapsulate the growth factor. Hence, the polysaccharides have to be converted into water-soluble small molecules to encapsulate growth factors easily. The procedure can be done by using acidic or enzymatic methods. In the acidic method, polysaccharides are dissolved in a 2-30% acidic solution of any kinds such as lactic acid, fruit acid, citric acid, acetic acid, hydrochloric acid, or ascorbic acid. Then, the acidic solution containing polysaccharides is stirred and the pH is adjusted to 2.7-3.5 so that long-chain large molecules are converted into water-soluble polysaccharides. Also, long-chain large polysaccharide molecules can be converted into water-soluble polysaccharides by cellulases. Hence, in the present invention, long-chain large polysaccharide molecules can be converted using acidic or enzymatic methods. Furthermore, the pH 4.6-6 polysaccharide solution (containing water-soluble small polysaccharide molecules) provided in the present invention can be a small molecule polysaccharide solution processed by acidic or enzymatic methods with a polysaccharide concentration by weight of 0.001-3%.

In step (B) mentioned above, the pH value of the polysaccharide solution is adjusted by an alkaline solution. The alkaline solution may be NaOH, KOH, ammonia solution, phosphate, or a combination of these, preferably NaOH. The volume of the alkaline solution is about 1-5% of the total volume. Furthermore, the pH of the polysaccharide solution can be adjusted by titrating the alkaline solution at a rate of 10-15 ml/min. Moreover, when the growth factor is added in step (B), the growth factor and the polysaccharide are stirred uniformly at a rate of 450-650 rpm, preferably at 500 rpm for 15 minutes, and then stirred at 150-250 rpm, preferably at 200 rpm, to adjust the pH of the solution and simultaneously encapsulating the growth factor with the polysaccharide. Hence, the polysaccharide particle encapsulating the growth factor of the present invention is formed and the uniform size of the micro-particle can be controlled.

In the method described above, all polysaccharide solutions can be used for the present invention. Desirable polysaccharide solutions may be chitin, chitosan, mucopolysaccharide, cellulose, starch, peptidoglycan, or a combination of any of these. chitosan, mucopolysaccharide, or a combination any of these are more desirable.

In addition, growth factory may be epidermal growth factor (EGF), recombinant human epidermal growth factor (rhEGF), fibroblast growth factor (FGF), and other acidic growth factors in the method described above. The acidic growth factors refer to those that have an acidic isoelectric point. More desirable growth factors include fibroblast growth factor (EGF), recombinant human epidermal growth factor (rhEGF), or a combination of any of these.

The size of the polysaccharide micro-particle formed in the method described above can be of any size, preferably less than 3,000 nm. More desirable size range is 1-1,000 nm; the size range of 1-500 nm is even better and the best size range is 30-200 nm to achieve the best efficacy for healing wounds.

On the other hand, with regard to growth factors with an alkaline isoelectric point, the present invention provides another method for producing a polysaccharide micro-particle to encapsulate the growth factor, which is similar to the method described above where the polysaccharide encapsulates the growth factor through electrostatic interaction. This method comprises the following steps: (A) providing a pH 2-4 polysaccharide solution and a pH 9-12 alkaline solution containing growth factors; and (B) adding the polysaccharide solution to the alkaline solution, and adjusting the pH of the alkaline solution to 6-8, preferably6.5-7.5, to obtain the polysaccharide micro-particle encapsulating the growth factor by electrostatic interaction. In this method, the percentage by weight of the polysaccharide solution is 0.1-3% of the total weight.

In the second method, the polysaccharide solution is added to the alkaline solution containing growth factors at a rate of 10-15 ml/min in step (B) to adjust the pH of the polysaccharide solution. As the solution containing growth factors is alkaline, adding the polysaccharide solution slowly to the alkaline solution containing growth factors can adjust the pH of the solution. Sufficient mixing of the growth factor and the polysaccharide solution can be achieved by stirring the mixture at a rate of 150-250 rpm, preferably at 200 rpm so that the polysaccharide has sufficient time to encapsulate the growth factor through electrostatic interaction to form the polysaccharide micro-particle encapsulating the growth factor according to the present invention.

The type of growth factors, alkaline solution, polysaccharide, and the size of the polysaccharide micro-particle required by the second method are similar to those used for the encapsulation of growth factors with an acidic isoelectric point. Thus, we shall not elaborate details here.

In addition, the present invention also provides a pharmaceutical composition for healing wounds comprising of a polysaccharide micro-particle encapsulating a growth factor and a pharmaceutically acceptable carrier. The polysaccharide micro-particle encapsulating a growth factor include one or more growth factors and a polysaccharide shell that can form a space to encapsulate the growth factor by electrostatic interaction and conformational change. As the growth factor in the present invention can promote cell division, activation, and proliferation, the growth factory can be used to heal wounds such as diabetic wounds, bums, mucotisis, and corneal ulcer. More importantly, the polysaccharide micro-particle can promote cell proliferation and reduce the possibility of the degradation of growth factor by proteinases in wounds or skin. Plus, the positive charge of the polysaccharide can further help the growth factor to get closer to the target cell, and increase the possibility of the growth factor coming in contact with the receptor. As a result, the growth factor encapsulated by the polysaccharide micro-particle in the present invention is more effectively for those that are not encapsulated in both in vitro and in vivo. More importantly, the growth factor encapsulated by polysaccharide according to the present invention can be kept efficacious for 2 years in solution form. This can reduce production costs and facilitate the commercialization of growth factors. Thus, the present invention overcomes conventional limits that growth factors cannot be commercialized.

As the pharmaceutical composition for healing wounds according to the present invention is the same as the polysaccharide particle encapsulating the growth factor in the method mentioned above, we shall not elaborate the details again. The pharmaceutically acceptable carrier of the pharmaceutical composition for healing wounds in the present invention is not limited to specific types, but is preferably one that is selected from activators, adjuvants, dispersants, humectants, and suspending agents. For example, the carrier can be water, gel, hyaluronic acid, natural oils, or glucose.

The polysaccharide micro-particle encapsulating the growth factor formed according to the present invention can be produced easily at low costs. In addition, to as the polysaccharide is non-toxic, biocompatible and biodegradable the bio-acceptability of the polysaccharide micro-particle of the present invention is increased greatly. Furthermore, the polysaccharide micro-particle of the present invention can improve the permeability of skin and blood capillaries, and prevent the degradation of proteinases in wounds. Thus, the possibility of the action of growth factor on fibroblasts is increased. Moreover, the storage life of the preparations containing the growth factor can be prolonged to at least 2 years. Even if the preparations are stored at room temperature for 2 years, the growth factor can still possess an activity rate of 87%. In other words, the encapsulation overcomes conventional limit that growth factors must be stored at −20° C. and increases the storage life. The present invention allows growth factors to be stored at room temperature for a long period of time.

Other advantages, and innovative features of the invention will be illustrated in the following detailed description with accompanying diagrams.

### BRIEF DESCRIPTION OF THE DIAGRAMS

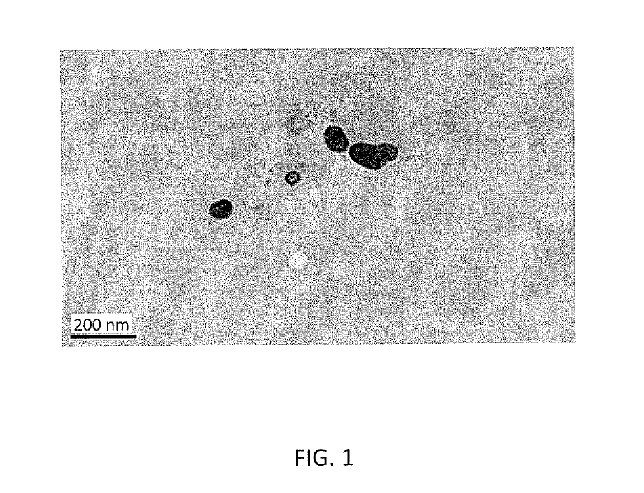

FIG. 1 is TEM of Example 1 of the present invention;

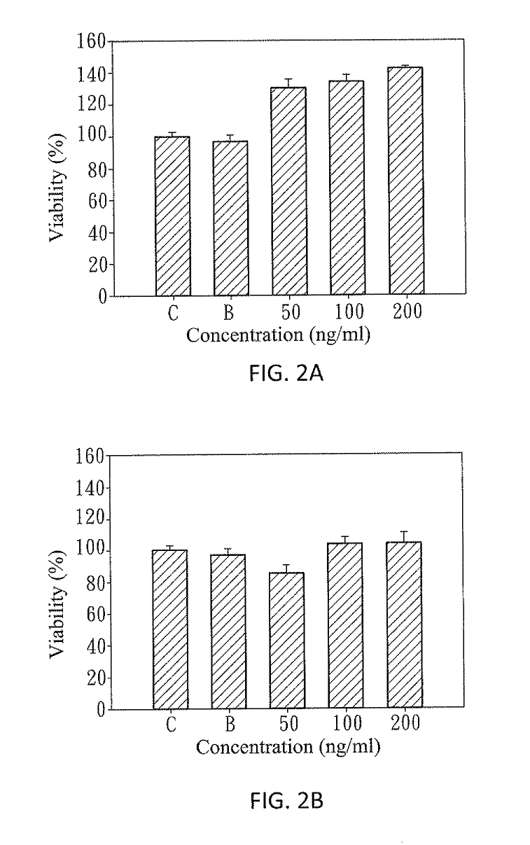

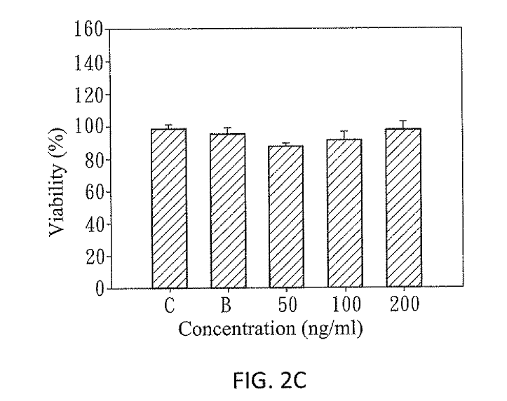

FIG. 2A shows the result that rhEGF encapsulated by the polysaccharide micro-particle is applied in NIH 3T3 cell survival test of Example 3 of the present invention;

FIG. 2B shows the result that the growth factor not encapsulated by the polysaccharide micro-particle is applied in NIH 3T3 cell survival test of Example 3 of the present invention; and

FIG. 2C shows the result that another growth factor not encapsulated by the polysaccharide micro-particle is applied in NIH 3T3 cell survival test of Example 3 of the present invention.

### DETAILED DESCRIPTION OF THE PREFERRED EMBODIMENT

Below are specific embodiments illustrating the practices of the present invention. Those familiar with this technique can easily understand other advantages and benefits of the present invention through the content disclosed. The present invention can also be practiced or applied by other variant embodiments. Possible modifications and variations based on different outlooks and applications can be made without departing from the core spirit of the invention.

### EXAMPLE 1

### Preparation of Polysaccharide Micro-Particles Encapsulating Growth Factors

In this example, 2% by weight of chitosan powder is first added to water. The mixture is stirred at 200 rpm for 5-10 mins. Then, the aqueous solution containing 2% by weight of chitosan is added to 10-30% v/v HCl solution, stirring uniformly. The mixture is then filtrated to remove impurities. The solution is adjusted to pH 2.7-3.5, forming an acidic chitosan solution.

A 1.5-10% v/v NaOH solution is used as the alkaline solution of the present invention to adjust the pH of the acidic chitosan solution processed by the acidic method. Then, the pH of the acidic chitosan solution is adjusted to more than 4.6 by titrating the alkaline solution at a rate of 20 ml/min. Recombinant human Epidermal growth factor (rhEGF) is then added to the adjusted solution until the final concentration of rhEGF reached 1-100 μg/ml or an optimal concentration of 4-10 μg/ml. In the chitosan solution with pH greater than 4.6, the rhEGF is negatively charged and water-soluble small chitosan molecule is positively charged. Subsequently, the solution is stirred at 500 rpm for 15 mins so that rhEGF is mixed uniformly in the chitosan solution with pH greater than 4.6.

Then, the chitosan solution containing rhEGF is stirred at 200 rpm and its pH is adjusted to 7.0 by titrating 1.5-10% v/v NaOH solution at a rate of 12 ml/min. During the titration process, positively charged short-chain small chitosan molecules will encapsulate the negatively charged rhEGF, and changed its own conformation with the increase in pH value, forming the chitosan micro-particles encapsulating the rhEGF in the present example. Finally, the solution is stirred at 500 rpm for 15 mins, and centrifuged at 12000-15000 rpm for 20 mins. The supernatant is discarded to obtain the hydrogel chitosan micro-particles encapsulating the rhEGF. The chitosan micro-particles made according to the present example are shown in the TEM effect diagram in FIG. 1. FIG. 1 shows the chitosan micro-particles encapsulating the rhEGF are spheroids. Through particle-size analysis, the size of the chitosan micro-particles range from 30-200 nm and the main size range of micro-particles varies from 50-100 nm.

### EXAMPLE 2

### Stability Analysis of the Chitosan Micro-Particles Encapsulating rhEGF

The stability analysis of chitosan micro-particles encapsulating rhEGF in this example includes a specific activity test and microbial test. The specific activity test adopts the Balb/c3T3 cells and international standard of epidermal growth factor (EGF) as the testing system. The MTT colorimetry is utilized to obtain the bioactivity (IU/ml) of the test sample. The bioactivity was divided by the protein amount (mg/ml) obtained by Lowry protein assay to obtain the specific activity of the sample (IU/mg). The microbial test is performed according to the “INSPECTIONS OF ASPECTIC PROCESSING” of pharmacopoeias.

First, the chitosan micro-particles encapsulating the rhEGF in Example 1 are divided into three sample groups: sample group 1 (0.109 mg/mi), sample group 2 (0.103 mg/ml), and sample group 3 (0.118 mg/ml). These three sample groups are stored at 2-8° C. chiller, 25° C. temperature-controlled chamber, and 37° C. chamber respectively. The samples are observed for 24 months and tested at 0, 6, 12, 18, and 24 months. The results are shown in Table 1 to 3.

TABLE 1
Sample Group 1

| | Storage | | Specific | |
|---|---|---|---|---|
| | life | Storage | Activity | Microbial |
| Test Date | (month) | Temperature | (IU/mg) | Test |
| 2007 Jan. 10 | 0 | — | 0.52 × 106 | Pass |
| 2007 Jul. 7 | 6 | 2-8° C. | 0.51 × 106 | Pass |
| | | 25° C. | 0.49 × 106 | Pass |
| | | 37° C. | 0.48 × 106 | Pass |
| 2008 Jan. 15 | 12 | 2-8° C. | 0.54 × 106 | Pass |
| | | 25° C. | 0.51 × 106 | Pass |
| | | 37° C. | 0.50 × 106 | Pass |
| 2008 Jul. 9 | 18 | 2-8° C. | 0.51 × 106 | Pass |
| | | 25° C. | 0.49 × 106 | Pass |
| | | 37° C. | 0.47 × 106 | Pass |
| 2009 Jan. 21 | 24 | 2-8° C. | 0.46 × 106 | Pass |
| | | 25° C. | 0.45 × 106 | Pass |
| | | 37° C. | 0.43 × 106 | Pass |

TABLE 2
Sample Group 2

| | Storage | | Specific | |
|---|---|---|---|---|
| | life | Storage | Activity | Microbial |
| Test Date | (month) | Temperature | (IU/mg) | Test |
| 2007 Jan. 10 | 0 | — | 0.50 × 106 | Pass |
| 2007 Jul. 7 | 6 | 2-8° C. | 0.51 × 106 | Pass |
| | | 25° C. | 0.50 × 106 | Pass |
| | | 37° C. | 0.49 × 106 | Pass |
| 2008 Jan. 15 | 12 | 2-8° C. | 0.59 × 106 | Pass |
| | | 25° C. | 0.51 × 106 | Pass |
| | | 37° C. | 0.52 × 106 | Pass |
| 2008 Jul. 9 | 18 | 2-8° C. | 0.54 × 106 | Pass |
| | | 25° C. | 0.52 × 106 | Pass |
| | | 37° C. | 0.50 × 106 | Pass |
| 2009 Jan. 21 | 24 | 2-8° C. | 0.50 × 106 | Pass |
| | | 25° C. | 0.51 × 106 | Pass |
| | | 37° C. | 0.44 × 106 | Pass |

TABLE 3
Sample Group 3

| | Storage | | Specific | |
|---|---|---|---|---|
| | life | Storage | Activity | Microbial |
| Test Date | (month) | Temperature | (IU/mg) | Test |
| 2007 Jan. 10 | 0 | — | 0.52 × 106 | Pass |
| 2007 Jul. 7 | 6 | 2-8° C. | 0.51 × 106 | Pass |
| | | 25° C. | 0.49 × 106 | Pass |
| | | 37° C. | 0.47 × 106 | Pass |
| 2008 Jan. 15 | 12 | 2-8° C. | 0.57 × 106 | Pass |
| | | 25° C. | 0.54 × 106 | Pass |
| | | 37° C. | 0.53 × 106 | Pass |
| 2008 Jul. 9 | 18 | 2-8° C. | 0.53 × 106 | Pass |
| | | 25° C. | 0.52 × 106 | Pass |
| | | 37° C. | 0.51 × 106 | Pass |
| 2009 Jan. 21 | 24 | 2-8° C. | 0.53 × 106 | Pass |
| | | 25° C. | 0.51 × 106 | Pass |
| | | 37° C. | 0.46 × 106 | Pass |

The results in table 1 to 3 show that all three sample groups passed the microbial test. Thus, variance errors that may influence the test results are excluded. In addition, the specific activity of samples stored at 2-8° C. and 25° C. for 24 months, and at 37° C. for 18 months did not decrease significantly. Although the specific activity of the samples stored at 37° C. for 24 months decreases slightly, the result is still within the acceptable range. These results prove that the chitosan micro-particles encapsulating the rhEGF in Example 1 can be stored at 2-8° C., 25° C. (room temperature), and 37° C. for at least 2 years. This shows that the chitosan micro-particles encapsulating the rhEGF in Example 1 possess a much longer storage life than that of those processed by conventional methods. Furthermore, the present invention overcomes the limits of conventional storage temperature and increases the storage life of growth factors at 25° C. (room temperature), and 37° C.

In addition to the temperature conditions mentioned above, the chitosan micro-particles encapsulating the rhEGF were heated at 100° C. for 30 mins and then analyzed by ELISA for their activity. The results are shown in Table 4. In Table 4, Sample 1 is not heated and Sample 2 is heated at 100° C. for 30 mins. 0.1 g of both sample 1 and 2 are mixed with the reagent (10 ml, including 1% BSA in PBS, pH 7.2-7.4) separately. 25 μl of each mixture (is mixed again with the reagent (10 ml, including 1% BSA in PBS, pH 7.2-7.4). Finally, 100 μl of the supernatant of each mixture is taken o ELISA analysis. The results in Table 4 show that sample 2 which was heated at 100° C. for 30 mins still possess at least 60% of original activity. This means polysaccharides are able to efficiently protect growth factors from high temperature damages.

TABLE 4

| | | Detected | Analysis |
|---|---|---|---|
| Sample | Unit | Value | Method |
| 1 | μg/ml | 8.77 | ELISA |
| 2 | μg/ml | 5.59 | ELISA |

### EXAMPLE 3

### The Influence of of Pharmaceutical Composition for Healing Wounds on the Viability of Fibroblasts

Fibroblasts are found all over an organism and can secrete collagen which is an important factor in wound healing. In this example, NIH 3T3 fibroblasts are used to prove the efficacy of the pharmaceutical composition of the present invention in healing wounds.

First, the samples are divided into three groups: Group 1, Group 2, and Group 3. All three groups are further divided into a control group (B), a blank group (C), and experimental subgroups. The experimental subgroups of Group 1 are chitosan micro-particles encapsulating rhEGF at 50 ng/ml, 100 ng/ml, and 200 ng/ml. NIH 3T3 cells are cultured using DMEM in a 96-well microplate at 37° C. and 5% CO₂. Then, the DMEM is removed and DMEM without blood serum is added. In blank group (C), no reagents are added. In control group (B), the solvent used for the preparation of growth factor is added. In the experimental subgroups, the chitosan micro-particles encapsulating the rhEGF at 50 ng/ml, 100 ng/ml, and 200 ng/ml are added respectively. The groups are analyzed by MTT cell viability assay after being cultured at 37° C. and 5% CO₂. The results are shown in FIG. 2. FIG. 2A shows the viability of NIH 3T3 cells which were cultured with chitosan micro-particles encapsulating thr rhEGF. FIG. 2B shows the viability of NIH 3T3 cells which were cultured with growth factors not encapsulated by chitosan. FIG. 2C shows the viability of NIH 3T3 cells which were cultured with another growth factor not encapsulated by chitosan, and the growth factor was used within a month after being prepared. In FIG. 2A, the viability of NIH 3T3 cells cultured with the chitosan micro-particles encapsulating the rhEGF is 40% higher than the blank group (C) and control group (B), and is dose-dependent. Thus, the chitosan micro-particles encapsulating rhEGF in the present invention can stimulate fibroblast proliferation. On the contrary, FIGS. 2B and 2C show that growth factors not encapsulated by chitosan and were used within a month after being prepared are unable to stimulate fibroblast proliferation. Furthermore, if the chitosan micro-particles encapsulating rhEGF of the present invention is applied on burns for 7 days, is observed that wounds are healed and scar proliferation is reduced significantly, thus, reducing the hospitalization time. It is observed that chronic diabetic wounds heal significantly after 6 weeks after applying the chitosan micro-particles of the present invention. This example shows that the chitosan micro-particles encapsulating rhEGF of the present invention possess outstanding efficacy in healing wounds and produce better results than growth factors not encapsulated by polysaccharides.

The above-mentioned examples are described only for a convenient illustration. The scope claimed in the present invention should be based on the claim listing hereinafter but is not restricted by the examples described above.

What is claimed is:

1. A polysaccharide micro-particle encapsulating one or more growth factors, comprising:
the growth factor; and
a polysaccharide shell forming a space to encapsulate the growth factor by electrostatic interaction.

2. The polysaccharide micro-particle of claim 1, wherein the growth factor is at least one selected from a group consisting of epidermal growth factor (EGF), recombinant human epidermal growth factor (rhEGF), fibroblast growth factor (FGF), and platelet-derived growth factor (PDGF).

3. The polysaccharide micro-particle of claim 1, wherein the polysaccharide shell is made of at least one selected from a group consisting of chitin, chitosan, mucopolysaccharide, cellulose, starch, and peptidoglycan.

4. The polysaccharide micro-particle of claim 1, which is in size of 30-3,000 nm.

5. A method for manufacturing polysaccharide micro-particles encapsulating one or more growth factors, comprising the following steps:
(A) providing a pH 4.6-6 polysaccharide solution and the growth factor; and
(B) adding the growth factor to the polysaccharide solution and adjusting the pH of the polysaccharide solution to 6-8 to obtain the polysaccharide micro-particle encapsulating the growth factor by electrostatic interaction.

6. The method of claim 5, wherein the polysaccharide solution contains 0.1-0.3% by weight of one ore more polysaccharides in the total polysaccharide solution.

7. The method of claim 6, wherein the polysaccharide is at least one selected from a group consisting of chitin, chitosan, mucopolysaccharide, cellulose, starch, and peptidoglycan.

8. The method of claim 5, wherein the growth factor is at least one selected from a group consisting of epidermal growth factor (EGF), recombinant human epidermal growth factor (rhEGF), fibroblast growth factor (FGF), and platelet-derived growth factor (PDGF).

9. The method of claim 5, wherein in step (B), the pH value of the polysaccharide solution is adjusted by an alkaline solution and the alkaline solution contains NaOH, KOH, ammonia, phosphate, or a combination thereof with 1-15% concentration by volume of the total alkaline solution.

10. The method of claim 9, wherein in step (B), the pH of the polysaccharide solution is adjusted by titrating the alkaline solution at a rate of 10-15 ml/min.
